(19) United States
(12) Patent Application Publication
(10) Pub. No.: US 2004/0236415 A1
(43) Pub. Date: Nov. 25, 2004

(54) Medical devices having drug releasing polymer reservoirs

(76) Inventor: Richard Thomas, Cloverdale, CA (US)

Correspondence Address: MEDTRONIC VASCULAR, INC., IP LEGAL DEPARTMENT, 3576 UNOCAL PLACE, SANTA ROSA, CA, 95403, US

(21) Appl. No.: 10/749,186

(22) Filed: Dec. 30, 2003

Related U.S. Application Data
(60) Provisional application No. 60/437,801, filed on Jan. 2, 2003.

(51) Int. Cl. A61F 2/06

(52) U.S. Cl. 623/1.42

(57) ABSTRACT

An improved implantable prosthesis for delivering one or more drugs to a target site within a lumen of a patient is disclosed. The implantable prosthesis includes a stent with a drug-releasing reservoir in the form of a sheath or strand of material. The device is generally configured to effectively and efficiently treat coronary artery disease. In addition, the device and treatment methods reduce patient recovery times and hospital costs and overall improve the quality of life for patients.

### RELATED APPLICATIONS

The present application claims priority to U.S. Provisional Patent Application Ser. No. 60/437,801 filed Jan. 2, 2003, the entire contents of which are incorporated herein by reference.

### FIELD OF THE INVENTION

The present invention relates to implantable medical devices having drug eluting reservoirs. Specifically, the present invention relates to vascular stents having drug eluting reservoirs made form polymeric materials.

### BACKGROUND OF THE INVENTION

Each year, thousands of people with coronary artery disease need treatment to increase the blood flow to their hearts. Although a variety of treatment options currently exist, treatment depends on many factors, such as a person's age, heart muscle function, the size and location of the arterial obstruction, and other health issues. Typical treatment options for coronary artery disease include drug therapy, balloon angioplasty, coronary artery bypass grafting and coronary stents.

Balloon angioplasty and coronary stents are two treatment options specifically designed to treat the complications resulting from artherosclerosis and other forms of coronary vessel narrowing. In general, angioplasty involves enlargement of the affected coronary artery lumen by radial expansion. The procedure is accomplished by maneuvering a first guidewire, which is about 0.038 inches in diameter, through the vascular system and to the site of therapy. A guiding catheter is then advanced over the first guidewire and positioned at a point just proximal to the stenosis. The first guidewire is removed and a second guidewire, having a balloon catheter mounted thereon, is advanced within the guiding catheter to a point just proximal of the stenosis.

The second guidewire is advanced into the stenosis, followed by the balloon on the distal end of the catheter. The balloon is then inflated within the narrowed lumen of the vessel causing the site of the stenosis to widen. Radial expansion of the vessel occurs in several different dimensions related to the nature of the occlusion or plaque. For example, soft, fatty plaque deposits are flattened by the balloon, whereas hardened plaque deposits are cracked and split to enlarge the vessel lumen. In addition, the wall of the vessel itself is also stretched when the balloon is inflated.

Dilatation of the occlusion, however, can also form flaps, fissures and dissections which may threaten reclosure of the dilated vessel or even perforations in the vessel wall. As such, implantation of a stent can provide the necessary support for such flaps and dissections and thereby prevent reclosure of the vessel. Alternatively, the stent may also function as a repair patch for a perforated vessel wall until corrective surgery can be performed. In general, a stent is a miniature expandable mesh tube made of medical grade stainless steel or other biomedical alloy. Examples of conventional stents include those disclosed in U.S. Pat. No. 4,733,665 issued to Palmaz, U.S. Pat. No. 4,886,062 issued to Wiktor, or U.S. Pat. No. 5,292,331 issued to Boneau which are incorporated herein by reference in their entirety.

U.S. Pat. No. 6,015,432 (the '432 patent) discloses a tubular structure that consist of a textile or other polymeric material and through which is threaded a supereleastic alloy such as nitinol. In one embodiment the wire or textile can be coated with a therapeutic agent. However, the '432 patent does not disclose dispersing the therapeutic agent within the textile or polymeric material so as to act as a drug reservoir.

The stent, which is generally pre-mounted on a deflated balloon catheter, is delivered to the affected area of the vessel using standard catheterization techniques, similar to those previously described. Once the catheter is positioned across the target area, the balloon catheter is inflated to circumferentially expand the stent and satisfactorily enlarge the lumen of the vessel. With the stent fully expanded into position within the lumen, the balloon is then deflated and the delivery device withdrawn, leaving the stent in the vessel lumen. Depending on the type and length of blockage, it may be necessary to place more than one stent in the vessel. Within time, the inside lining of the vessel eventually heals around the stent which functions as a miniature “scaffolding” to provide the necessary support to maintain the vessel in an open position.

Although stents are generally effective at treating coronary artery disease and vessel occlusion, some drawbacks have been encountered with practically all prior art stents. For example, in some instances and despite the presence of the stent, the vessel restenoses or forms new blockages at the site of stent placement. There are generally two mechanisms that cause or trigger restenosis. The first mechanism is thrombosis or blood clotting. The risk of thrombosis is greatest immediately after the angioplasty procedure because the resultant tissue trauma tends to trigger blood clotting. This form of restenosis is greatly reduced by using anticoagulant and antiplatelet drugs.

The second mechanism is tissue in-growth at the site of treatment or stent placement. This form of restenosis produces a proliferation of the endothelial cells that normally line blood vessels. However, unlike thrombosis, the resultant tissue in-growth or scar-like formation within the vessel lumen is not systemically treatable with anticoagulant and/or antiplatelet drugs. In general, this form of restenosis requires a small amount of a drug that inhibits tissue growth to be delivered directly to the site of tissue in-growth.

In view of the above, there is a need for an improved device for effectively and efficiently treating coronary artery disease. In particular, it is desirable that the device has a high success rate at treating coronary artery disease with minimal to no side-effects or related complications. The device should include improved drug delivery capabilities, such as the ability to deliver one or more drugs directly to a treatment site. In addition, the device and treatment methods should reduce patient recovery times and hospital costs and overall improve the quality of life for patients.

### BRIEF SUMMARY OF THE INVENTION

The present invention provides implantable medical devices having a therapeutic agent delivery reservoir associated therewith. The reservoir is a polymer, or polymer blend and may be composed from natural polymers (biomolecules) or synthetic polymers (bioresobable/biodegradable and bio-stable/non-biodegradable). The therapeutic agent polymer reservoir may be composed of single stands of polymer woven throughout the implantable medical devices either longitudinally or horizontally. In the alternative the therapeutic reservoirs may be in the form of a sleeve, wrapping, covering or sheath (collectively a sheath). The sheath may be woven from single stands of polymer or extruded or milled.

The implantable medical devices of the present invention include, but are not limited to vascular stents, vascular grafts and endovascular support devices useful in treating stenoses, restenoses, aneurysms and other structural defects associated with body lumens including blood vessels and secretory ducts.

In view of the foregoing, it is an object of the present invention to provide an improved device for effectively and efficiently treating coronary artery disease.

It is a further object of the present invention to provide a device having a high success rate at treating coronary artery disease with minimal to no side-effects or related complications.

It is a further object of the present invention to provide a device having improved drug delivery capabilities, such as the ability to deliver one or more drugs directly to a treatment site.

A further object of the present invention is to provide a device and treatment methods that reduce patient recovery times and hospital costs and overall improve the quality of life for patients.

For example, and not intended as a limitation, one embodiment of the present invention provides an implantable medical device having a thin-walled tubular member having a plurality of openings and at least one elongated polymer strand woven through the openings wherein the elongated polymer strand has incorporated therein or thereon at least one therapeutic agent for release into tissue adjacent the elongated polymer strand when the implantable medical device is implanted into a vessel.

In another related embodiment of the present invention a method for providing a therapeutic agent to tissue in need thereof includes providing an implantable medical device having a thin-walled tubular member having a plurality of openings and at least one elongated polymer strand woven through the openings wherein the elongated polymer strand has incorporated therein or thereon at least one therapeutic agent for release into the tissue and deploying the implantable medical device to the tissue in need of a therapeutic agent.

### BRIEF DESCRIPTION OF THE DRAWINGS

Other features and advantages of the present invention will be seen as the following description of particular embodiments progresses in conjunction with the drawings, in which:

FIG. 1 is perspective view of an embodiment of a stent in accordance with the present invention;

FIGS. 2A and 2B illustrate an embodiment of a stent surrounded by a drug delivery sheath in accordance with the present invention;

FIGS. 3A and 3B illustrate an alternate embodiment of a stent surrounded by a drug delivery sheath in accordance with the present invention;

FIG. 4 is a sectional view of an embodiment of a stent surrounded by a plurality of drug delivery sheaths in accordance with the present invention;

FIG. 5 illustrates an alternate embodiment of a stent surrounded by a plurality of drug delivery sheaths in accordance with the present invention;

FIGS. 6A and 6B illustrate perspective views of a stent including one or more drug-loaded strands of material in accordance with the present invention; and

FIGS. 7A and 7B illustrate perspective views of an embodiment of a stent including one or more drug-loaded strands of material in accordance with the present invention.

### DETAILED DESCRIPTION OF THE INVENTION

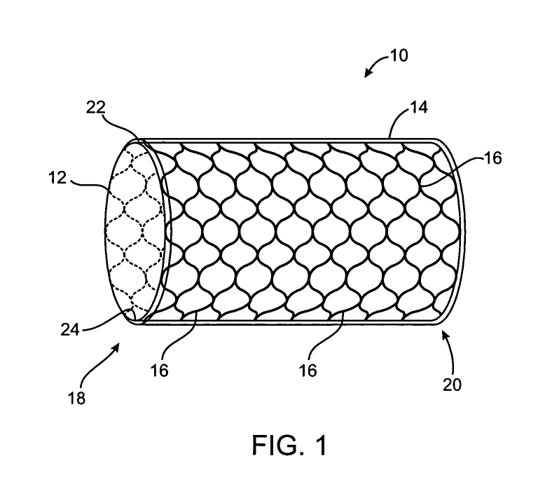

Referring to FIG. 1, an embodiment of an implantable prosthesis 10 in accordance with the present invention includes a stent 12 with a drug-releasing reservoir 14. In the spirit of convenience and brevity, the implantable prosthesis 10 referenced in the text and figures of the present disclosure is a stent. However, it should be noted that other implantable prostheses 10 including, but not limited to, vascular grafts, endovascular support devices, catheters, or other implantable devices are also within the scope of the claimed invention.

The illustrative stent 12 shown in FIG. 1 includes a geometrical arrangement of one or more wire filaments 16 that form the framework for the tubular-shaped device. The filaments 16 are configured to permit the stent 12 to be compressed and expanded in axial and/or radial directions, while still maintaining sufficient mechanical force when implanted so as to prevent vessel restenosis or collapse. While one embodiment of the stent 12 includes wire filaments 16, it is understood that the present invention is applicable to all known stent constructions, such as welded wire, chemical etching, laser etching, laser fusion, annealing, shaping, rings, electropolishing and other stent constructions known to those skilled in the art. Furthermore, the stent 12 depicted in FIG. 1 can be expandable or self-expanding. Expandable stents are generally deployed as discussed above whereby the stent is first placed over the distal tip of a catheter having an expandable balloon integrated into the catherter's distal end. In this embodiment the stent is compressed, or “crimped” onto the catheter prior to deployment. In one embodiment of the present invention the filament of sheath containing the therapeutic agent is crimped over the balloon together with the stent.

The tubular shaped stent 12 forms a lumen having a first end 18, a second end 20, an external vessel-contacting surface 22 and an internal surface 24. The internal surface 24 defines the internal diameter of the stent 12, which is sized to accommodate unrestricted blood-flow through the vessel (not shown) and is generally within the range of approximately 1.5 to 7 mm (0.059 to 0.276 inch) in its expanded state. As with stent diameter, the length of the stent 12, or the distance between the first end 18 and the second end 20, is determined in part by the size of the vessel and/or target area into which the stent 12 is to be implanted. In general, the stent 12 is preferably of sufficient length as to maintain its axial orientation without shifting under the hydraulics of fluid flow within the vessel. In one embodiment, the length of the stent 12 is approximately within the range of 8 to 40 mm (0.315 to 1.57 inch) in its expanded state and is generally configured to extend across at least a significant portion of the target area (not shown).

In order for the stent 12 to be either permanently or temporarily implanted within the lumen of a patient, the stent 12 is preferably constructed of biocompatible materials having sufficient mechanical strength and durability. In one embodiment of the invention, the stent 12 is fabricated from medical grade stainless steel. Alternate materials including, but not limited to, nitinol, Titanium, tantalum, cobalt-based alloys, bioresorbable materials, ceramics, plastics, composites, and polymers. In general, the polymer chosen for stent fabrication must be a polymer that is biocompatible and minimizes irritation to the vessel wall when the medical device is implanted. The polymer may be either a biostable (non-biodegradable) or a bioabsorbable (biodegradable) polymer depending on the desired rate of release or the desired degree of polymer stability. Bioabsorbable polymers that could be used include poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid.

Also, biostable polymers with a relatively low chronic tissue response such as polyurethanes, silicones, and polyesters could be used and other polymers could also be used if they can be dissolved and cured or polymerized on the medical device such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

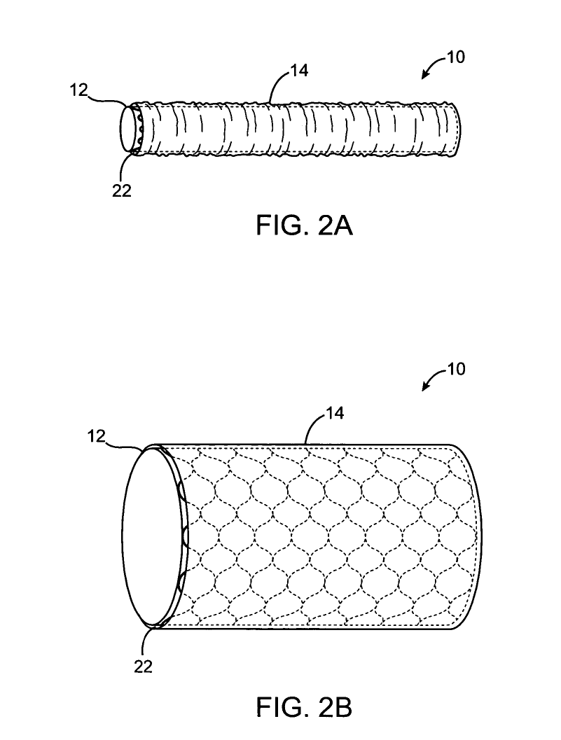

As noted in the Background of the Invention set forth above, some medical procedures and/or conditions require site-specific treatment utilizing drugs. As the stent 12 of the present invention provides a preferred means with which to deliver such drugs, it is instructive to describe the elements or components that form the drug dispensing stent 12. For this purpose, reference is made to FIGS. 2A and 2B.

FIGS. 2A and 2B illustrate one embodiment of the present invention wherein the stent 12 is covered with a drug delivery sleeve or sheath 14 comprising a material impregnated with one or more drugs. The term “drug,” “therapeutic” and/or “bioactive agent” as used herein means any compound intended for use in animals having a desired effect. Non-limiting examples include anticoagulants, such as an RGD peptide-containing compound, heparin, antithrombin compounds, platelet receptor antagonists, anti-thrombin antibodies, anti-platelet receptor antibodies, aspirin, protaglandin inhibitors, platelet inhibitors, or tick anti-platelet peptide. Other classes of drugs includes vascular cell antiproliferative agents, such as a growth factor inhibitor, growth factor receptor antagonists, transcriptional repressor or translational repressor, antisense DNA, antisense RNA, replication inhibitor, inhibitory antibodies, antibodies directed against growth factors, cytotoxic agents, cytoskeleton inhibitors, peroxisome proliferator-activated receptor gamma (PPARγ) agonists, molecular chaperone inhibitors and bifunctional molecules. The drug can also include cholesterol-lowering agents, vasodilating agents, and agents which interfere with endogenous vasoactive mechanisms. Other examples of drugs can include anti-inflammatory agents, anti-platelet or fibrinolytic agents, anti-neoplastic agents, anti-allergic agents, anti-rejection agents, metalloprotease inhibitors, anti-microbial or anti-bacterial or anti-viral agents, hormones, vasoactive substances, anti-invasive factors, anti-cancer drugs, antibodies and lymphokines, anti-angiogenic agents, radioactive agents and gene therapy drugs, among others.

Specific non-limiting examples of drugs that fall under one or more of the above categories include paclitaxel, docetaxel and derivatives, epothilones, nitric oxide release agents, heparin, aspirin, coumadin, D-phenylalanyl-prolyl-arginine chloromethylketone (PPACK), hirudin, polypeptide from angiostatin and endostatin, benzoquinone ansamycins including geldanamycin, herbimycin and macbecin, methotrexate, 5-fluorouracil, estradiol, P-selectin Glycoprotein ligand-1 chimera, abciximab, exochelin, eleutherobin and sarcodictyin, fludarabine, sirolimus, rapamycin, tetrazole-containing immunosuppressant macrolide antibiotics (for example Abbott Laboratories ABT-578. See, for example U.S. Pat. No. 6,015,815. Specifically, Examples 1, 1A and 2 for synthesis and claims 1, 2 and 3 for structures, all of which are incorporated herein by reference), certican, Sulindac, tranilast, thiazolidinediones including rosiglitazone, troglitazone, pioglitazone, darglitazone and englitazone, tetracycline antibiotics (tetracyclines), VEGF, transforming growth factor (TGF)-beta, insulin-like growth factor (IGF), platelet derived growth factor (PDGF), fibroblast growth factor (FGF), RGD peptide, estrogens including 17 beta-estradiol, metalloprotease inhibitors and beta or gamma ray emitter (radioactive) agents.

As shown in FIG. 2A, when the stent 12 is in an unexpanded or collapsed state, the drug delivery sheath 14 is configured to loosely surround the stent 12. In this regard, the sheath 14 may be folded, pleated, twisted, crimped, wrapped or similarly gathered around the external surface 22 of the stent 12. In general, the sheath 14 should be configured to at least partially envelop the stent 12 so as to provide a low profile that facilitates device delivery (e.g., via a catheter) and deployment/expansion within the lumen of the patient. As used herein a “sheath” may be either woven from individual polymeric stands, extruded as a single intact sheet or tube, as in the case of polytetrafluoroethylene (PTFE AKA Teflon®) and similar polymers or milled from a solid polymer into a sleeve or sheath. Moreover, as used herein “sleeve” is synonymous with sheath.

When the device 10 is in an expanded state, the sheath 14 forms a barrier or covering over at least a portion of the external surface 22 of the stent 12. As shown in FIG. 2B, stent expansion causes the sheath 14 to unfold and compresses the sheath 14 against the lumen of the patient (not shown). The outwardly extending radial force exerted by the stent 12 on the sheath 14 and lumen prevents the stent 12 and/or sheath 14 from becoming dislodged or migrating away from the target site. In addition, contact between the drug-loaded sheath 14 and the wall of the lumen causes the drug(s) to be released from the sheath 14 and absorbed by the tissue at the desired target site.

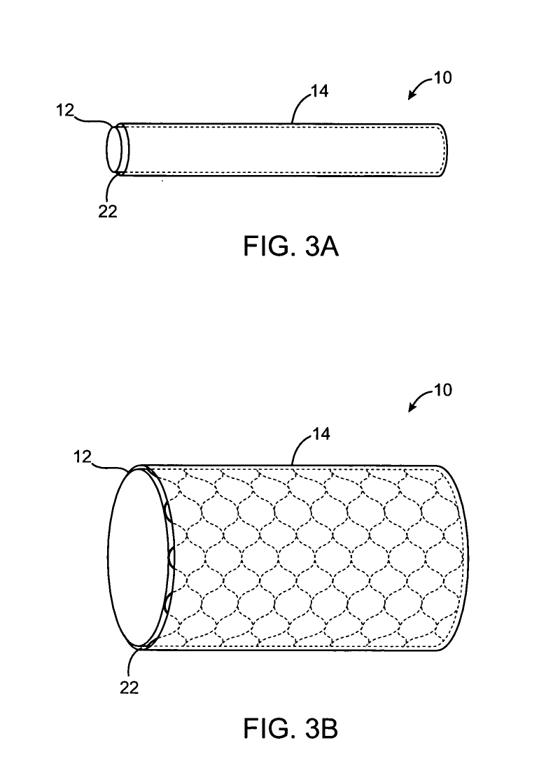

In an alternate embodiment of the invention, the drug delivery sheath 14 is fabricated from an elastic-type material having expansion and compression characteristics similar to those of the stent 12. As shown in FIGS. 3A and 3B, the sheath 14 substantially conforms to the shape of the stent 12 in both its unexpanded and expanded states. In some instances, when the fibers or elements comprising the sheath material expand to accommodate the shape of the implanted stent 12, not only do the fibers elongate but the spaces or pores between the fibers also increase is size. As such, fluids such as blood, systemically-delivered drugs, activator agents, and other fluids known to those skilled in the art flow through the lumen and pores of the device 10 saturating both the device 10 and the target tissue. This device configuration is thought to provide improved fluid flow through the walls of the device 10 and to the tissue target site, which may also produce enhanced therapeutic and diagnostic capabilities.

For example, in one embodiment of the invention, the sheath 14 may be impregnated with an agent-activated drug. During use, the device 10 is implanted within the lumen of a patient following conventional stent delivery techniques. As the stent 12 is deployed, it expands and compresses the drug-loaded sheath 14 against the tissue wall of the lumen. However, the drug(s) are not released from the device 10 until they are activated by their compatible agent(s). The drug activating agents are typically introduced into the blood flow of the patient and, upon contacting the stent 12, trigger a controlled release of the drug(s) from the sheath 14.

This particular device configuration provides greater control over the volume/amount of drug(s) administered to the target site and the timing by which the drug(s) are released. As such, a wide variety of drugs and release agents may be used in combination with the device 10 of the present invention for various treatment/diagnostic procedures. For example, a full dosage of a release agent may be administered to the patient during a single procedure for treatment/diagnosis of a particular condition. Alternatively, partial dosages of release agents may be administered to the patient during multiple procedures and over a more prolonged period of time (e.g., minutes, hours, days, weeks, months, etc.), thereby allowing for a more controlled method of treatment/diagnosis tailored to the specific needs of each patient. As such, a variety of conditions may be treated and/or diagnosed. Further, enhanced site-specific treatment/diagnosis may also be accomplished when the device is configured to include multiple drugs at specific locations on the sheath 14 and used in combination with a variety of drug-compatible release agents.

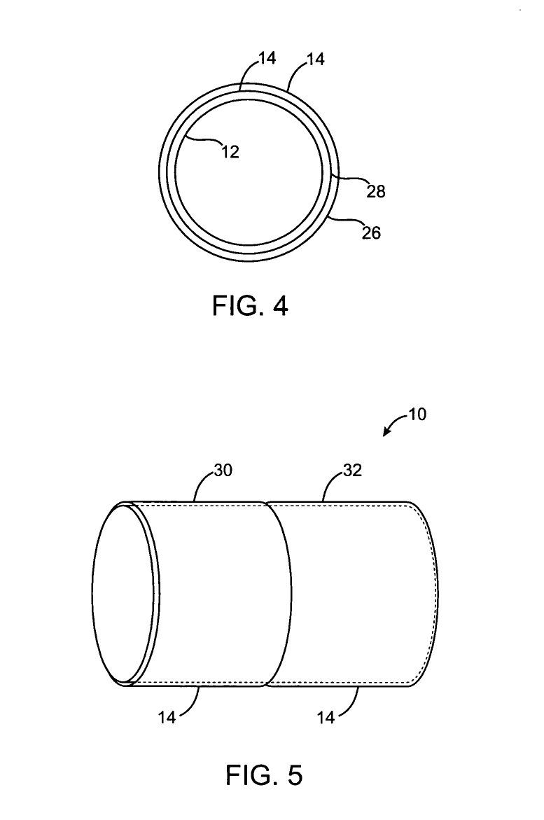

In an alternate embodiment of the invention, more than one sheath 14 may be applied to a stent 12. As shown in FIG. 4, two drug-loaded sheaths 14 are concentrically aligned on a stent 12. Although only two sheaths 14 are illustrated, it is understood that multiple sheaths 14 may be used and are included within the scope of the claimed invention. This device configuration provides an alternate means of controlling drug delivery via the sheath layers. For example, the outer sheath 26 may be fabricated from a resorbable material that, over time, provides structural support when implanted within the patient's lumen. Once the outer sheath 26 is resorbed, the inner sheath 28 may be activated to deliver a drug which prevents tissue in-growth and restenosis. In an alternate example, the sheaths 26, 28 may be impregnated with various drugs that are to be delivered to the tissue target site in substantially a sequential manner or phased release. As such, after the drug(s) from the outer sheath 26 are absorbed by the tissue, the drug(s) from the inner sheath 28 are subsequently absorbed by the tissue target site.

Referring to FIG. 5, an alternate embodiment of a multi-sheath device 10 includes two drug-loaded sheaths 14 aligned along the longitudinal axis of the stent 12. Although only two sheaths 14 are illustrated, it is understood that multiple sheaths 14 may be used and are included within the scope of the claimed invention. This device configuration provides yet another means by which drug delivery may be controlled and tailored to the specific needs of the patient. In particular, this device configuration allows site-specific treatment at multiple locations within the lumen. For example, the distal sheath 30 of the stent 12 may be impregnated with an antibiotic and the proximal sheath 32 of the stent 12 may be impregnated with a steroid.

As is evident from the previously described embodiments, the drug-loaded sheath 14 may be secured to the stent 12 via friction and/or compression forces. In an alternate embodiment (not shown), the sheath(s) 14 may be secured to the stent 12 via hooks, adhesives, welds, chemical bonds, stitches. In general, the sheath(s) 14 should be sufficiently secured onto the stent 12 to prevent stent migration within or dislodgement from the target site within the lumen.

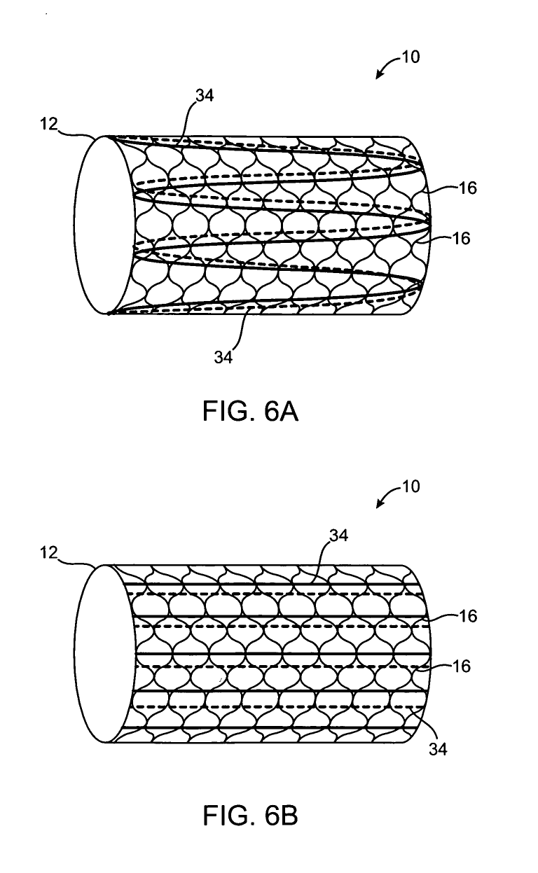

In an alternate embodiment of the invention, one or more strands or threads 34 of material are woven through the filaments 16 of the stent 12. As shown in FIG. 6A, an individual strand 34 of material may be woven through the filaments 16 along the longitudinal axis of the stent 12 in a repeating pattern that also extends along the circumference of the device 10. Alternatively, multiple strands 34 of material may be individually woven through the filaments 16 and along the longitudinal axis of the device 10. As shown in FIG. 6B, in addition to their longitudinal arrangement, each strand 34 is also placed adjacent to the other strands 34 along the circumference of the stent 12.

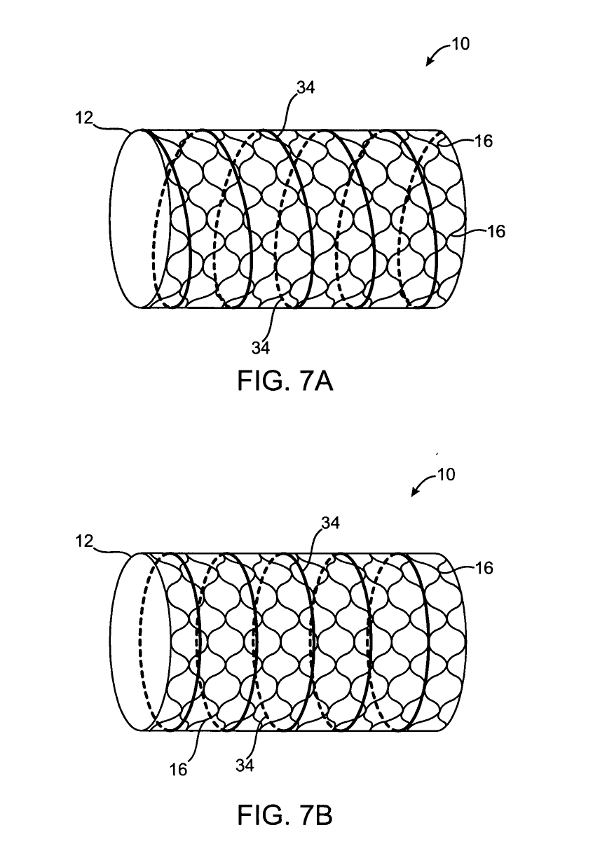

FIGS. 7A and 7B illustrate alternate embodiments wherein either a single or multiple strand(s) 34 are woven through the filaments 16 along the circumference/radius of the device 10 and extending along the stent's longitudinal axis. Alternate weave patterns that extend over at least a portion of the stent 12, not specifically disclosed herein but known to those skilled in the art, are also included within the scope of the claimed invention.

In general, the strands of material 23 are woven onto the stent 12 in order to securely attach the material onto the stent 12 in a manner that does not interfere with device deployment. As with the above-referenced sheaths 14, the strand(s) of material may also be loaded with one or more drugs and incorporated onto the stent 12 in various patterns and combinations for site-specific treatment and/or diagnosis.

The drug delivery sheath 14 of the present invention, whether formed as a continuous sleeve 14 or individual strands 34, may be fabricated from one or more materials that are biocompatible, non-toxic and capable of delivering drugs to a target site. The sheath/strand material and its structure should also be configured to allow fluids/blood to flow through the wall of the sheath/strand 14, 34. This design feature not only allows fluids to contact the tissue areas adjacent the device 10 but also prevents side branch occlusion in the event that the device 10 is deployed at or near a vessel side branch.

It is also desirable that the sheath/strand material prevents or mitigates any adverse, chronic local response when implanted within the lumen of the patient. In one embodiment, the drug-impregnated material that covers the stent 12 may be of a type that, after a period of time, is broken down by the body and absorbed into the body's tissue. Alternatively, bioresorbable materials (e.g., materials that decompose into water and carbon dioxide via hydrolysis) having drug-releasing capabilities may also be used to cover the stent 12 and, thereby, provide additional structural support to the lumen.

Examples of sheath/strand materials that may be used with the device of the present invention include, but are not limited to, resorbable polymers, synthetic polymers, natural polymers including fibrin, fibrinogens, starches and collagens, polyglycolic acid (PGA), poly(L-lactic acid) (PLLA), polydioxanone (PDS), poly(D,L-lactic acid) (PDLLA), polycaprolactone, poly(lactide-co-glycolide), poly(hydroxybutyrate), poly(hydroxybutyrate-co-valerate), polyorthoester, polyanhydride, poly(glycolic acid-co-trimethylene carbonate), polyphosphoester, polyphosphoester urethane, poly(amino acids), cyanoacrylates, poly(trimethylene carbonate), poly(immunocarbonate), copoly(ether-esters) (e.g., PEO/PLA), polyalkylene oxalates, polyphosphazenes, copolymers, tyrosine-derived polycarbonates, tricalcium phosphates, celluloses, hyaluronic acids, gels, proteins, allografts, hydrogels, PTFE (Polytetrafluoroethylene), Vicryl® (manufactured by Ethicon, New Jersey) Prolenee (manufactured by Ethicon, New Jersey), Mersilene® (manufactured by Ethicon, New Jersey), polyethylene fiber, and GORE-TEX® (manufactured by W. L. Gore & Associates, Arizona). In addition, biostable polymers with a relatively low chronic tissue response such as polyurethanes, silicones, polyesters, polyolefins, polyisobutylene and ethylene-alphaolefin copolymers, acrylic polymers and copolymers, vinyl halide polymers and copolymers, such as polyvinyl chloride; polyvinyl ethers, such as polyvinyl methyl ether, polyvinylidene halides, such as polyvinylidene fluoride and polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose and other materials, including combinations thereof, known by those skilled in the art may also be used and are also included within the scope of the claimed invention.

In addition, the material(s) comprising the sheath(s) 14 and/or strand(s) 34 should also readily accept, retain and deliver one or more drugs to a target site within the lumen of a patient. As such, the material functions as a reservoir for improved drug-loading capabilities and controlled time-release of drugs. It is well known in the art how to incorporate one or more bioactive agent into a polymer and control the release therefrom. See for example co-pending U.S. patent application having attorney docket number 14364-0074, specifically paragraphs 69 through 110, the entire contents of which are incorporated herein by reference in their entirety.

Other treatment and/or diagnostic procedures utilizing various combinations of sheaths 14, sheath designs, strands 34, strand designs, drugs, release agents and medical procedures with the device 10 of the present invention, not disclosed herein but known to those skilled in the art, are also included within the scope of the claimed invention. As such, the device 10 and methods of the present invention provide for controlled drug release rates, localized drug delivery, long-term treatment and/or diagnostic capabilities. In addition, the device 10 and associated methods of the present invention as referenced above provide increased efficiency, therapeutic/diagnostic effectiveness, cost effectiveness and user convenience.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. An implantable medical device comprising:
a thin-walled tubular member having a plurality of openings and at least one elongated polymer strand woven through said openings wherein said elongated polymer strand has incorporated therein or thereon at least one therapeutic agent for release into tissue adjacent said elongated polymer strand when said implantable medical device is implanted into a vessel.

2. The medical device according to claim 1 wherein said implantable medical device is selected from the group consisting of a vascular stent, vascular grafts, endovascular support devices, and catheters.

3. The medical device according to claim 1 wherein said and at least one elongated polymer strand is woven through said openings longitudinally.

4. The medical device according to claim 1 wherein said and at least one elongated polymer strand is woven through said openings horizontally.

5. The medical device according to claim 1 wherein said and at least one elongated polymer strand comprises a biodegradable polymer.

6. The medical device according to claim 1 wherein said and at least one elongated polymer strand comprises a non-biodegradable polymer.

7. The medical device according to claim 1 wherein said and at least one elongated polymer strand comprises a biomolecule.

8. The medical device according to claim 5 wherein said biodegradable polymer is selected from the group consisting of poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) polyalkylene oxalates, polyphosphazenes and combinations thereof.

9. The medical device according to claim 6 wherein said non-biodegradable polymer is selected from the group consisting of polyurethanes, silicones, polyolefins, polyisobutylene and ethylene-alphaolefin copolymers; acrylic polymers and copolymers, ethylene-co-vinylacetate, polybutylmethacrylate, vinyl halide polymers and copolymers, polyvinyl ethers, polyvinyl methyl ether; polyvinylidene halides, polyacrylonitrile, polyvinyl ketones; polyvinyl aromatics, polyvinyl esters, copolymers of vinyl monomers, ethylene-methyl methacrylate copolymers, acrylonitrile-styrene copolymers, ABS resins, ethylene-vinyl acetate copolymers, polycarbonates, polyoxymethylenes, polyimides; polyethers, epoxy resins, polyurethanes, rayon, rayon-triacetate; cellulose, cellulose acetate, cellulose butyrate; cellulose acetate butyrate; cellophane; cellulose nitrate; cellulose propionate; cellulose ethers, and carboxymethyl cellulose, PTFE and combinations thereof.

10. The medical device according to claim 7 wherein said biomolecule is selected from the group consisting of fibrin, fibrinogen, cellulose, starch, collagen, hyaluronic acid and combinations thereof.

11. The medical device according to claim 1 wherein said therapeutic agent is selected from the group consisting of paclitaxel, docetaxel and derivatives, epothilones, nitric oxide release agents, heparin, aspirin, coumadin, D-phenylalanyl-prolyl-arginine chloromethylketone (PPACK), hirudin, polypeptide from angiostatin and endostatin, benzoquinone ansamycins including geldanamycin, herbimycin and macbecin, methotrexate, 5-fluorouracil, estradiol, P-selectin Glycoprotein ligand-1 chimera, abciximab, exochelin, eleutherobin and sarcodictyin, fludarabine, sirolimus, rapamycin, ABT-578, certican, Sulindac, tranilast, thiazolidinediones including rosiglitazone, troglitazone, pioglitazone, darglitazone and englitazone, tetracyclines, VEGF, transforming growth factor (TGF)-beta, insulin-like growth factor (IGF), platelet derived growth factor (PDGF), fibroblast growth factor (FGF), RGD peptide, estrogens, 17 beta-estradiol, metalloprotease inhibitors, beta or gamma ray emitter (radioactive) agents and combinations thereof.

12. The medical device according to claim 2 wherein said stent is crimped onto a balloon.

13. The medical device according to claim 2 wherein said stent in self expanding.

14. A method for providing a therapeutic agent to tissue in need thereof comprising:
providing an implantable medical device comprising:
a thin-walled tubular member having a plurality of openings and at least one elongated polymer strand woven through said openings wherein said elongated polymer strand has incorporated therein or thereon at least one therapeutic agent for release into said tissue;
deploying said implantable medical device to said tissue in need of a therapeutic agent.

15. The method according to claim 14 wherein said tissue in deploying step comprises a vessel lumen.

16. The method according to claim 14 wherein said implantable medical device in said proving step is a vascular stent.

17. A vascular stent comprising a thin-walled tubular member having a plurality of openings and at least one elongated polymer strand woven through said openings wherein said at least one elongated polymer strand has incorporated therein paclitaxel.

18. A vascular stent comprising a thin-walled tubular member having a plurality of openings and at least one elongated polymer strand woven through said openings wherein said at least one elongated polymer strand has incorporated therein rapamycin.

19. A vascular stent comprising a thin-walled tubular member having a plurality of openings and at least one elongated polymer strand woven through said openings wherein said at least one elongated polymer strand has incorporated therein a tetrazole-containing immunosuppressant macrolide antibiotic.

20. The vascular stent according to any one of claims 17, 18 or 19 wherein said polymer is selected from the group consisting of biomolecules, biodegradable polymers and non-biodegradable polymers.

21. The vascular stent according to claim 20 wherein said biomolecule is selected from the group consisting of fibrin, fibrinogen, cellulose, starch, collagen, hyaluronic acid and combinations thereof.

22. The vascular stent according to claim 21 wherein said biomolecule is fibrin.

23. A vascular graft comprising a thin-walled tubular member having a plurality of openings and at least one elongated polymer strand woven through said openings wherein said at least one elongated polymer strand has incorporated therein a therapeutic selected from the group consisting of paclitaxel, rapamycin, ABT-578 and and metalloprotease inhibitors.

24. An endovascular support device comprising a thin-walled tubular member having a plurality of openings and at least one elongated polymer strand woven through said openings wherein said at least one elongated polymer strand has incorporated therein a therapeutic selected from the group consisting of paclitaxel, rapamycin, ABT-578 and metalloprotease inhibitors.

25. An implantable medical device according to claim 1 or 14 where in said at least one elongated polymer strand forms a sheath surrounding at least a portion of said external surface, wherein said sheath comprises a material impregnated with one or more drugs.

26. An implantable medical device according to claim 25 wherein said sheath is formed from an extruded polymer.
